(19) United States
(12) Patent Application Publication
(10) Pub. No.: US 2007/0299418 A1
(43) Pub. Date: Dec. 27, 2007

(54) Fastening means in the form of a belt for an absorbent article

(75) Inventor: Kent Vartiainen, Lerum (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 11/819,899

(22) Filed: Jun. 29, 2007

Related U.S. Application Data
(63) Continuation of application No. PCT/SE04/02033, filed on Dec. 29, 2004.

(51) Int. Cl. A61F 13/15

(52) U.S. Cl. 604/392

(57) ABSTRACT

A belt fastener for an absorbent article (1) includes at least one belt portion (9a, 9b, 201), wherein the belt portions (9a, 9b, 201) have at least one nonwoven layer. At least one nonwoven layer in the belt portion is a composite material which has synthetic continuous filaments and natural fibers.

### TECHNICAL FIELD

The present invention relates to a fastener for an absorbent article in the form of a belt, which comprises at least one belt portion. The belt portion comprises at least one non-woven layer of composite material.

### BACKGROUND

Fasteners such as belts give a user of an absorbent article good potential for adjustment of the article, both during and after application. Fastening takes place in two steps. The first step is when the belt is fastened about the wearer's waist, the second when the absorbent member is fastened to the belt, so that the article takes a pant-like form. Belt-diapers also provide caregivers, such as hospital personnel, the potential to minimize strained movements while applying the absorbent article.

However, a series of disadvantages exist with belt-diapers, and particularly with the belts. The belt is most often manufactured with a laminate structure, to provide a sufficient tensile strength and to give the belt sufficient stiffness. The laminate structure is most often produced from nonwoven material which had been ultrasonically welded, thermo-bonded or glued together. The manufacturing process is complicated and requires a plurality of manufacturing steps, and is also costly.

Known laminate belts are made of synthetic fibers. This implies that the belts are most-commonly hydrophobic. Sweating often occurs when a user lies in a bed, e.g., at night-time. It has been shown that known laminate belts cause uncomfortable sensations, as sweat can gather as a thin layer between the users trunk and the belt of the diaper. This becomes especially marked when the bonding area in a laminate belt is high. Under e.g., ultrasound welding, the bonding points have a structure such that it is difficult for liquid to penetrate the belt at these points. The problem becomes even more noticeable when there are a number of bonding points which are relatively small, but tightly spaced. Small amounts of moisture can be formed over a large surface between the belt and the skin of the user, leading to the skin being experienced as damp or wet.

### SUMMARY

A fastener for an absorbent article of the type discussed in the introduction has been achieved which significantly minimizes the problems of previously-known fasteners. In one embodiment according to the invention, the fastener is in the form of a belt comprising at least one belt portion which comprises at least one nonwoven layer. The at least one belt portion comprising nonwoven is a composite material comprising synthetic continuous filaments, e.g., spunlaid, and natural fibers, e.g., cellulose fibers. The natural fibers are suitably hydrophilic. Said layer is preferably hydroentangled. Here, and in the following, “entangled” means to tangle or wound together. Here, and in the following, “user” means the individual wearing the diaper.

The fastener comprises a belt for a belt-diaper. To function as a belt, the fastener should exhibit a sufficiently high tensile strength, at the same time as being soft to the touch and having absorbing properties. Such a belt is not just user-friendly, as has just been described, but is also advantageously produced from a process technique point of view. In one embodiment of the invention, the belt can be at least partly formed of only one layer. This means that lamination of many materials as previously required to provide a belt, is not required. The belt is also relatively cheap to produce in comparison to other previously-known laminate belts, without compromising on qualities such as softness and strength.

Preferably, said nonwoven layer of composite material forms the user-facing side of the belt. According to one aspect of the invention, at least one portion of the belt is only formed of said nonwoven layer of composite material.

The synthetic continuous filaments may be formed from, or alternatively comprise, polypropene, polyethene or polylactide, or other suitable polymers.

The nonwoven layer of composite material according to an embodiment of the invention may further comprise synthetic staple fibers. The staple fibers exhibit a length of 1-15 mm, preferably 3-7 mm, and a thickness of circa 2-20 dtex. Long staple fibers help make the belt stiffer, while shorter staple fibers give an increased feel of textile material, which can be desirably in certain circumstances. The staple fibers can also be colored to give the belt a color other than that of the absorbent article.

Examples of suitable materials are described in international patent application ‘PCT/SE2004/001519 and in the Swedish patent application SE 0302874-3. According to an embodiment of the invention, the nonwoven layer of the belt comprises:
10-50% synthetic continuous filaments, preferably 15-35% synthetic continuous filaments
20-90% natural fibers, preferably 40-75% natural fibers,
0-50% synthetic staple fibers, preferably 5-25% synthetic staple fibers in weight percent based on the total weight of said nonwoven layer.

Of course, the various fiber contents cannot be selected such that the total amount exceeds 100%. In one embodiment, the fiber content is chosen such that the total amount of natural fiber and synthetic continuous filament in the belt's nonwoven layer exceeds 60%, preferably 70%, more preferably 80% and most preferably 90% in weight percent based on the total weight of said nonwoven layer of composite material.

When the nonwoven layer comprises 10% continuous filaments and 90% natural fibers, the belt gives high absorption capacity due to the high concentration of natural fibers. This can be advantageous on users who sweat a lot, such as for example upon use of the diaper in warm environments for longer periods. The belt gives a drier sensation against the skin as it sucks in the dampness which accumulates. However, a belt according to this embodiment is not as strong as an embodiment with a higher content of synthetic continuous filaments. 10% synthetic continuous filaments should therefore preferably be regarded as the minimum limit. With higher amounts of synthetic continuous filaments, the nonwoven layer becomes more expensive. To maintain a strong yet cheap nonwoven layer of composite material, it is therefore advantageous to keep within the range 10-50%, preferably 15-35%, synthetic continuous filaments in weight percent based on the total weight of said nonwoven layer. In another embodiment, in which the nonwoven layer comprises 50% continuous filaments and 50% hydrophilic fibers, a very strong nonwoven layer is obtained, which still shows absorbing properties. According to one embodiment of the invention, the belt comprises 10-50% synthetic staple fibers together with other fibers in the above-mentioned content ranges. The synthetic continuous filaments can for example exhibit a basis weight of 20-50 g/m². For the nonwoven layer of composite material, the basis weight is 50-150 g/m⁻², preferably 70-120 g/m².

The belt is preferably breathable. By “breathable”, it is meant that water vapour and other cases can pass through the belt.

### SHORT DESCRIPTION OF THE FIGURES

In the following, the invention shall be more closely described, with reference to the following figures.

Hereby:

FIG. 1 shows a belt diaper seen towards the liquid permeable topsheet

FIG. 2 shows a belt portion connected to an absorbent article.

### DESCRIPTION OF EMBODIMENTS

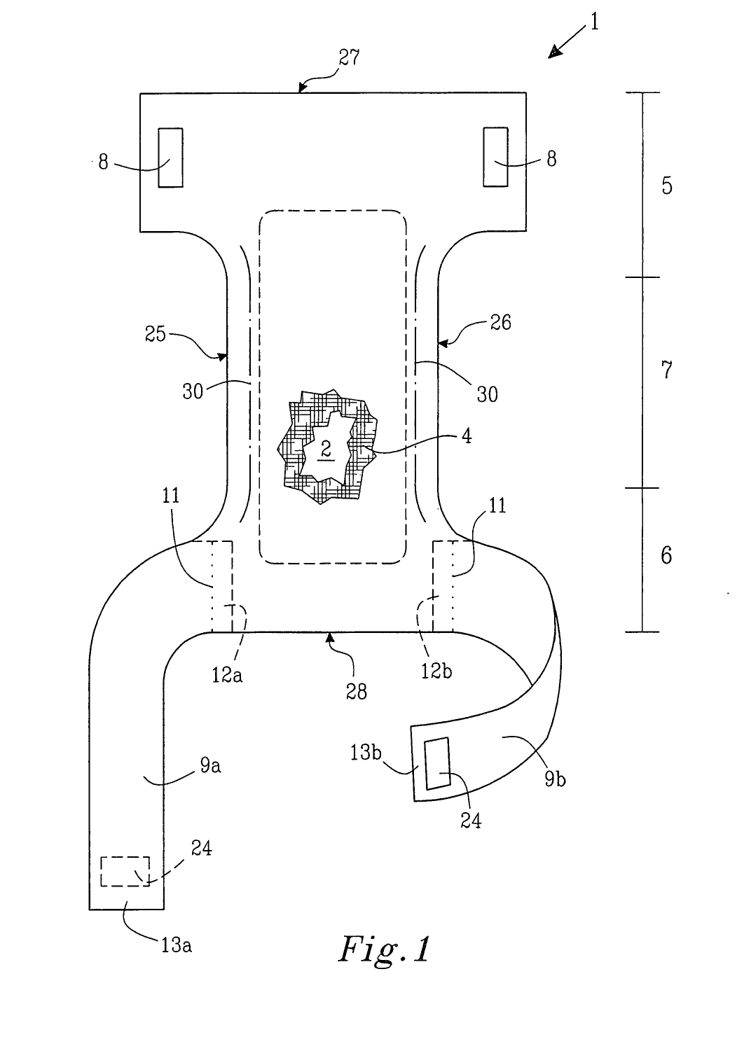

FIG. 1 shows an embodiment of an absorbent article in the form of a belt diaper 1 comprising a backsheet material 2 which should preferably be liquid impermeable, a liquid permeable topsheet 3 and an absorbent member 4 enclosed therebetween. The liquid permeable topsheet 3 can comprise nonwoven material, bonded carded fiber material, perforated plastic film or combinations thereof. In the case there the backsheet material is liquid impermeable, it may comprise material such as plastic film, nonwoven material which is coated with a liquid-blocking material or a hydrophobic nonwoven which resists liquid penetration. The backsheet material 2 is preferably breathable. By “breathable material” is meant a material which has the ability to allow water vapour and other gases through. Examples of such materials are well known in the field and will not be described further here.

The backsheet material 2 and the topsheet 3 have a somewhat larger planar extension than the absorbent member 4, and extend outside the edges of the absorbent member. The backsheet material 2 and the topsheet 3 are mutually connected within the projecting edges, e.g., though gluing or welding with heat or ultrasound. On each side of, and substantially parallel with, the absorbent member 4, run elastic elements 30. So-called “leakage barriers” (not shown) may also be fixed in the longitudinal direction of the absorbent member 4.

The absorbent member 4 can be of any conventional kind. Examples of normally occurring absorbent materials are cellulose fluff pulp, tissue layers, highly absorbing polymers (so-called “superabsorbents” or SAP), absorbent foam material, absorbent nonwoven material and the like. It is usual to combine cellulose fluff pulp with superabsorbents in an absorbent member. Absorbent members built up of layers of different material with different properties in respect of liquid receiving ability, liquid distribution ability and storage ability are also common. These are known to the person skilled in the art, and do not therefore need to be described in detail. The thin absorbent cores which are normal in e.g., baby diapers and incontinence shields often comprise a compressed, mixed or layered structure of cellulose fluff pulp and superabsorbents.

The belt diaper is intended to encompass the lower portion of the wearer's trunk like a pair of absorbent pants. It has a front part 5 which in use is intended to be turned forward on the wearer and a rear portion 6 which in use is intended to be turned backwards on the user. A groin portion 7 is located between the front and rear portions, which is use is intended to be placed in the wearer's groin, between their legs. The front portion 5 possesses a pair of front fastening members 8 of the hook and loop type. Adhesive fastening agents can also be used.

The belt 9 comprises a first and a second belt portion 9a, 9b. The belt portions are joined via a first end 12a, 12b along line 11 to the rear portion 6 of the diaper, for example by gluing or ultrasound welds. According to another embodiment, the belt portions can be formed from the same layer as the rear portion 6. The first and second belt portions 9a,9b are intended to be joined together through their other ends 13a, 13b with a belt fastening member 24. The belt fastening member 24 can be formed from hook and loop type material, e.g., VELCRO®, or from an adhesive fastening agent. The front fastening member 8 of the front portion 5 is intended to be joined to the outside of the belt portions 9a, 9b, in order to fasten the belt diaper to the desired pant-like form. According to an alternative embodiment (not shown), the first and the second belt portion 9a, 9b are fastened to the diaper's front portion 5 and are thereby intended to be joined together on the wearer's back. The fastening member 8 is thus arranged on the rear portion 6 of the diaper as a rear fastening member.

In an embodiment which is not shown, the backsheet 2 can be used as the front or rear fastening member 8.

To provide a comfortable fit, the belt portions 9a,9b should have a width of 5-20 cm, preferably 7-15 cm. The belt portions 9a, 9b can be formed from a nonwoven layer of composite material which comprises synthetic continuous filaments, e.g., spunlaid, and natural fibers such as cellulose fibers. Preferably, said layer is hydroentangled. The nonwoven layer of composite material can also be a part of a laminate, i.e., a support material, which can form at least a part of the belt. When the nonwoven layer of composite material forms a part of a laminate, the laminate can preferably comprise a nonwoven material which is formed from a carded, thermobonded material such as e.g., polypropylene, polyester or bi-component fibers. Such a layer can act as loop material for a hook and loop type fastening system.

A preferred embodiment is that where the nonwoven layer of composite material is not completely encompassed by the laminate, but only 50% or less of the area of the nonwoven layer is covered by another material, e.g., a carded, thermobonded material.

The nonwoven layer of composite material can be adapted to act as a reception surface for the front fastening member 8. In the case where the front fastening members are comprised of adhesive tape flaps, the reception zone is in the form of a suitable plastic film. In case other types of fastening members are used instead of adhesive tape flaps, e.g., hook and loop types, other types of material are appropriate which can function as a reception surface for the fastening member in question. Elastic laminates are also suitable for use as material in the belt portions 9a, 9b. An important detail is that the belt portions 9a, 9b are preferably made breathable so as not to occlude (i.e., obstruct) the skin of the user wearing it. Diaper 1 also shows two transverse end edges 27,28 as well as two longitudinal side edges 25, 26.

As shown in FIG. 1, the belt 9 in the belt-diaper can be formed from two belt portions 9a, 9b, but in accordance with an embodiment of the invention, the belt 9 can also be formed from only one belt portion.

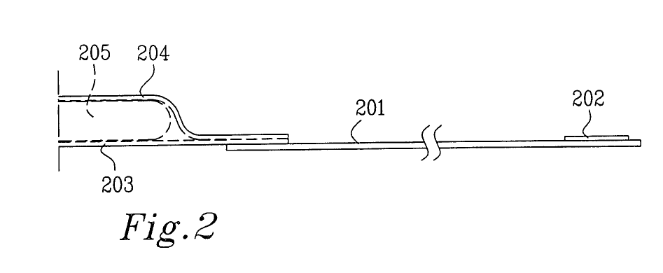

A belt portion 201 is shown in FIG. 2 which alone can form a belt, or can be used together with a corresponding belt portion, see FIG. 1. In use, the belt is fastened about the user's torso. The belt portion 201 has a means for fastening 202 in the form of a surface provided with a hook material which is arranged on belt portion 201. Belt portion 201 is fixedly arranged on a backsheet 203. A absorbent body 205 is placed between the backsheet 203 and the liquid permeable topsheet 204.

The belt portion 201 in FIG. 2 is made only of a composite nonwoven layer comprising 40% continuous filaments together with 60% cellulose fibers in weight percent based on the total weight of said nonwoven layer. The continuous filaments give tensile strength to the belt, while the cellulose fibers provide the belt with softness and the ability to absorb liquid. The amounts in the belt can be suited to give the belt varied properties within the scope of the invention.

Manufacture and Composition of the Composite Material

Filaments are fibers which are relatively long in proportion to their diameter; in principle they can in certain circumstances considered infinitely long relative to their diameter. They are produced through melting and extruding a thermoplastic polymer through small holes. The polymer is then cooled down, preferably by air being blown onto, and in line with the polymer threads. The polymer threads solidify, and can then be treated through being stretched, curled, crumpled or similar. Chemicals for surface treatment of the filaments can be applied to provide further properties of the filaments.

The filaments can also be manufactured through chemical reactions, via a solution with fiber-forming reagents which are placed in a reagent, e.g., through spinning viscose fibers from cellulose xanthate solution in sulphuric acid. So-called meltblown fibers are produced via extruding melted thermoplastic polymers through small holes so that fine threads are formed, at the same time as converging air streams are directed forwards the polymer threads. The polymer threads are drawn out to long continuous filaments with a small diameter. Manufacture of meltblown fibers is described in detail’ in U.S. Pat. No. 3,849,241 and U.S. Pat. No. 4,048,364. The fibers can be so-called microfibers or macrofibers depending on their dimensions. Microfibers usually have a diameter of <20 μm, usually 2-12 μm. Macrofibers have a diameter of over 20 μm, usually 21-100 μm.

So-called spunbond filaments are produced in a like manner as that described above, but the air streams have a lower temperature and the air streams stretch the filament to achieve a suitable diameter. The fiber diameter is usually over 10 μm, usually 10-110 μm. Production of spunbond is described for example in patent document U.S. Pat. No. 4,812,864 and U.S. Pat. No. 5,545,371.

As a group, spunbond and meltblown filaments are usually called “spun laid filaments”. This involves that they are laid directly on a moveable surface to make a layer which is bound together later in the process. In this way, the synthetic continuous filaments have a diameter of 2-100 μm, preferably 10-100 μm.

Thermoplastic polymers which have sufficiently coherent strength properties which allow them to be stretched out in their melted condition, as described above, can in principle be used to produce meltblown or spunbond filaments. Examples of polymers which can be used are: polyolefins, such as polyethene, polypropene, polyamide, polyester and polylactides. Copolymers of these polymers may also be used.

Many different natural fibers can be found which may be used in accordance with the invention. Fibers which have the capacity to absorb water and tend to help the formation of a cohesive layer are especially useful. The natural fibers are preferably hydrophilic. Examples of natural fibers are cellulose fibers, cotton fibers, hemp fibers, leaf fibers, jute fibers and the like. Preferred fibers are paper pulp fibers. Pulp fibers are preferably between 1-5 mm. Recycled pulp fibers may be used. Examples of paper pulp fibers are chemical pulps, CTMP and TMP. Staple fibers can be produced with the same material and same processes as mentioned earlier. Staple fibers can be treated with so-called “spin finish”, i.e., surface treated and curled or crumpled. Spin finishing is usually used to give the fibers a hydrophilic surface when they are used in e.g., the outer surface of diapers. As opposed to filaments, staple fibers are cut to suitable lengths. The lengths can vary depending on the purpose. When the staple fibers are used in hydroentangled nonwoven layers, the length is 1-15 mm, preferably 3-7 mm.

One way to produce a material according to the invention comprises the following steps:

First, a continuous material web is provided, upon which the continuous filaments can be laid down. Excess air is sucked through said material web so that a layer of filament is formed on the material web. The layer of continuous filaments is then moved to a wet-laying section, where a slurry comprising a mixture of natural fibers and water, possibly together with staple fibers as well, is wet-laid upon the layer of continuous filaments. Remaining water is sucked away through the material web. In one embodiment, the slurry containing a mixture of natural fibers and water, possibly together with staple fibers, is wet-laid on the layer of continuous filaments so that they at least partly penetrate into the layer of continuous filaments.

The layer of continuous filaments and the mixture of natural fibers are transported to an apparatus for hydroentangling. Here, the filaments and fibers are mixed, and bound together to a nonwoven layer by many small water jet streams being sprayed on the layer at high pressure.

The hydroentangled layer, i.e., the nonwoven layer, is transported to a drying stage where the layer is dried. The layer may be transported further for rolling, cutting and packing.

In accordance with the previously-described method, the continuous filaments are laid down directly on the continuous material web, where they form an unbonded layer. This unbonded layer can in this instance look like a net structure. By letting the filaments cool down sufficiently before they land on the material web, the layer keeps a certain amount of mobility. This is because, after cooling down, the filaments are not sticky. The speed at which the filaments are laid down on the material web is much higher than the speed of the material web. This means that the filaments form irregular loops and bends as they are laid on the material web. This contributes greatly to the irregularity that the filaments make in the layer. The basis weight of the layer formed with continuous filaments should be between 2-50 g/m².

Pulp fibers, and possibly also staple fibers, which can be added as required, are carefully blended to a slurry. Possible additives, such as wet or dry strength substances, retention agents, dispersing agents, pigment or mixtures thereof can be added. The mixture is pumped out via a headbox on the material web, where it then lies on the unbonded continuous filaments. Pulp fibers and staple fibers will mainly stay above the layer of continuous filaments. Only a few fibers will penetrate between the filaments. The excess water is sucked through the material web and the fiber layer with the help of a drying box which is placed under the material web.

The fiber web of continuous filament, pulp fiber and possibly staple fiber is hydroentangled while it still lies on the material web. Hydroentangling mixes the fibers intensively and binds the fibers together to a nonwoven of composite material. A full description of hydroentangling is described in patent CA 841 938 A.

In hydroentangling, the various fibers will become significantly homogeneously mixed and integrated with each other, and form a nonwoven of composite material. The finer mobile spunlaid filaments twist themselves together and build a strong material together with the other fibers. The energy which is required for entangling is relatively low, i.e., material of this type is easy to entangle. The energy which is put in to such an entangling is in the order of magnitude 50-500 kwh/ton.

In one embodiment, no binding—e.g., thermobonding or hydroentangling—of the continuous filaments is carried out before pulp fibers and possibly staple fibers are laid on the continuous filaments. The continuous filaments should preferably be free to move, which makes mixing, integration and binding together of the fibers easier under hydroentangling. With this step, possible thermobonded points would prevent pulp fibers and staple fibers from being caught up or entangled in the continuous filaments, as they would be immobilized by the bonding points. A two-layer material will be produced instead of a well-mixed and integrated composite material. The strength of a hydroentangled material based on pulp fibers or staple fibers or mixtures thereof depends mostly upon the number of bonding points, i.e., points where the fibers are entangled together. Long fibers are therefore preferable. When a filament is used, the strength of the material comes primarily from the filaments. The strength is also obtained relatively quickly upon hydroentangling. Therefore, the energy in the entangling process in used primarily for mixing the fibers and to maintain good integration of the fibers. The unbonded, open structure which the filaments have makes this integration easier.

The nonwoven layer of composite material is then dried through a conventional drying step.

While the present invention has been particularly shown and described with reference to exemplary embodiments thereof, it will be understood by those skilled in the art that various changes in form and details may be made therein without departing from the spirit and scope of the invention as defined by the following claims.

What is claimed is:

1. A belt fastener for an absorbent article, the belt fastener comprising at least one belt portion, wherein the at least one belt portion comprises at least one nonwoven layer that is a composite material which comprises synthetic continuous filaments and natural fibers.

2. The belt fastener for an absorbent article according to claim 1, wherein the natural fibers are hydrophilic fibers.

3. The belt fastener for an absorbent article according to claim 2, wherein the hydrophilic fibers are cellulose fibers.

4. The belt fastener for an absorbent article according to claim 1, wherein the synthetic continuous fibers are spunlaid.

5. The belt fastener for an absorbent article according to claim 1, wherein said nonwoven layer of composite material is hydroentangled.

6. The belt fastener for an absorbent article according to claim 1, wherein the synthetic continuous filaments comprise polypropene, polyethene or polylactide.

7. The belt fastener for an absorbent article according to claim 1, wherein said nonwoven layer of composite material further comprises synthetic staple fibers with a length of 1-15 mm.

8. The belt fastener for an absorbent article according to claim 1, wherein said nonwoven layer of composite material comprises:
10-50% synthetic continuous filaments;
20-90% natural fibers; and
0-50% synthetic staple fibers, in weight percent based on the total weight of said nonwoven layer.

9. The belt fastener for an absorbent article according to claim 1, wherein said nonwoven layer of composite material has a basis weight of 50-150 g/m².

10. The belt fastener for an absorbent article according to claim 1, wherein said nonwoven layer of composite material forms a user-facing side of the belt.

11. The belt fastener for an absorbent article according to claim 1, wherein at least one portion of the belt is only formed of said nonwoven layer of composite material.

12. The belt fastener for an absorbent article according to claim 1, wherein the total amount of natural fiber and synthetic continuous filament in the nonwoven layer of the belt exceeds 60% in weight percent based on the total weight of said nonwoven layer of composite material.

13. The belt fastener for an absorbent article according to claim 1, wherein said nonwoven layer of composite material further comprises synthetic staple fibers with a length of 3-7 mm.

14. The belt fastener for an absorbent article according to claim 1, wherein said nonwoven layer of composite material has a basis weight of 70-120 g/m².

15. The belt fastener for an absorbent article according to claim 1, wherein the total amount of natural fiber and synthetic continuous filament in the nonwoven layer of the belt exceeds 70% in weight percent based on the total weight of said nonwoven layer of composite material.

16. The belt fastener for an absorbent article according to claim 1, wherein the total amount of natural fiber and synthetic continuous filament in the nonwoven layer of the belt exceeds 80% in weight percent based on the total weight of said nonwoven layer of composite material.

17. The belt fastener for an absorbent article according to claim 1, wherein the total amount of natural fiber and synthetic continuous filament in the nonwoven layer of the belt exceeds 90% in weight percent based on the total weight of said nonwoven layer of composite material.
